(19) United States
(12) Patent Application Publication
(10) Pub. No.: US 2025/0360062 A1
(43) Pub. Date: Nov. 27, 2025

(54) COMPOSITION FOR CARING FOR AND/OR MAKING UP KERATIN MATERIALS

(71) Applicant: L'OREAL, Paris (FR)

(72) Inventors: Hongling XU, Shanghai (CN); Yangdong CHEN, Shanghai (CN)

(73) Assignee: L'OREAL, Paris (FR)

(21) Appl. No.: 18/872,480

(22) PCT Filed: Jun. 30, 2022
(86) PCT No.: PCT/CN2022/102821

§ 371 Date: Dec. 6, 2024

(51) Int. Cl. A61K 8/06; A61K 8/36; A61K 8/60; A61K 8/891; A61Q 1/00; A61Q 19/00

(52) U.S. Cl. CPC A61K 8/062; A61K 8/361; A61K 8/60; A61K 8/891; A61Q 1/00; A61Q 19/00

(57) ABSTRACT

It relates to a composition in the form of an oil-in-water emulsion for caring for and/or making up keratin materials, comprising: (i) relative to the total weight of the composition, at least 0.6 wt. % of at least one first surfactant selected from silicone surfactants; (ii) at least one second surfactant different from the first surfactant and different from saturated C14-C22 fatty acids; (iii) at least one structuring agent selected from saturated C14-C22 fatty acids; and (iv) at least one saponifier, wherein the weight ratio of the structuring agent to the second surfactant is from 0.2 to 2.0. It also relates to a non-therapeutic method for caring for keratin materials, comprising applying said composition to the skin.

### TECHNICAL FIELD

The present invention relates to a cosmetic composition. In particular, the present invention relates to a composition for caring for and/or making up keratin materials. The present invention also relates to a non-therapeutic method for caring for and/or making up keratin materials.

### BACKGROUND ART

The skin is the protective barrier for the human body. It protects the interior of the body from physical injury (such as trauma) and biological injury (such as bacteria, viruses or fungi).

The development of formulations dedicated to caring for and/or making up the skin and/or lips, is permanent.

For example, efforts have been made to formulate compositions in the form of creams or emulsions.

For some products, they will tend to foam when they are applied, which is unpleasant for consumers.

As a commercial product, it is desired that the product is stable at room temperature and even at a high temperature for example above 45° C.

However, for some emulsions such as water-in-oil emulsions or oil-in-water emulsions, phase separation is easy to occur.

Hence, there is a need to formulate a composition for caring for and/or making up the skin, which does not foam during application and is stable at a relatively high temperature for example 45° C.

### SUMMARY OF THE INVENTION

The inventors have now discovered that it is possible to formulate a composition for caring for the skin, which does not foam during application and is stable at a relatively high temperature for example 45° C.

Accordingly, according to a first aspect, the present invention provides a composition in the form of an oil-in-water emulsion for caring for and/or making up keratin materials, comprising:
(i) relative to the total weight of the composition, at least 0.6 wt. % of at least one first surfactant selected from silicone surfactants;
(ii) at least one second surfactant different from the first surfactant and different from saturated C14-C22 fatty acids;
(iii) at least one structuring agent selected from saturated C14-C22 fatty acids; and
(iv) at least one saponifier,
wherein the weight ratio of the structuring agent to the second surfactant is from 0.2 to 2.0.

The composition of the present invention is in the form of an oil-in-water emulsion. Thus, said composition comprises a continuous aqueous phase and a dispersed fatty phase.

The inventors found that the composition of the present invention has a lamellar structure or liquid crystals.

The composition of the present invention does not foam during application and is stable at a relatively high temperature for example 45° C.

According to a second aspect, the present invention provides a non-therapeutic method for caring for keratin materials, comprising applying the composition according to the first aspect of the present invention to the keratin materials.

Other advantages of the present invention will emerge more clearly on reading the description and the examples that follow.

### BRIEF DESCRIPTION OF THE DRAWINGS

Implementations of the present invention will now be described, by way of example only, with reference to the attached FIGURE, wherein:

FIG. 1 shows a photo of the composition of invention example 1 (IE.1) taken with a polarized light microscopy.

### DETAILED DESCRIPTION OF THE INVENTION

Unless otherwise defined, all technical and scientific terms used herein have the same meaning as commonly understood by those skilled in the art the present invention belongs to. When the definition of a term in the present description conflicts with the meaning as commonly understood by those skilled in the art the present invention belongs to, the definition described herein shall apply.

In that which follows and unless otherwise indicated, the limits of a range of values are included within this range, in particular in the expressions “between . . . and . . . ” and “ranging from . . . to . . . ”.

Moreover, the expression “at least one” used in the present description is equivalent to the expression “one or more”.

Throughout the instant application, the term “comprising” is to be interpreted as encompassing all specifically mentioned features as well as optional, additional, unspecified ones. As used herein, the use of the term “comprising” also discloses the embodiment wherein no features other than the specifically mentioned features are present (i.e. “consisting of”).

Unless otherwise specified, all numerical values expressing amount of ingredients and the like which are used in the description and claims are to be understood as being modified by the term “about”. Accordingly, unless indicated to the contrary, the numerical values and parameters described herein are approximate values which are capable of being changed according to the desired purpose as required.

For the purposes of the present invention, the term “keratin materials” is intended to cover human skin, mucous membranes such as the lips. Facial skin is most particularly considered according to the present invention.

All percentages in the present invention refer to weight percentage, unless otherwise specified.

According to the first aspect, the present invention provides a composition in the form of an oil-in-water emulsion for caring for and/or making up keratin materials, comprising:
(i) relative to the total weight of the composition, at least 0.6 wt. % of at least one first surfactant selected from silicone surfactants;
(ii) at least one second surfactant different from the first surfactant and different from saturated C14-C22 fatty acids;
(iii) at least one structuring agent selected from saturated C14-C22 fatty acids; and
(iv) at least one saponifier,
wherein the weight ratio of the structuring agent to the second surfactant is from 0.2 to 2.0.

### First Surfactant

According to the first aspect, the composition of the present invention comprises at least one first surfactant selected from silicone surfactants.

The term “silicone surfactant” means a silicone compound comprising at least one oxyalkylene chain. In a preferred embodiment the silicone surfactant comprises at least one oxyethylene (—OCH2CH2-) and/or oxypropylene (—OCH2CH2CH2-) chain.

These silicone surfactants may be chosen from nonionic, anionic, cationic and amphoteric silicone surfactants. Reference may be made to the document “Encyclopaedia of Chemical Technology, Kirk-Othmer”, volume 22, pp. 333-432, 3rd edition, 1979, Wiley, for the definition of the properties and (emulsifying) functions of surfactants, in particular pp. 347-377 of this reference, for anionic, amphoteric and nonionic surfactants.

The silicone surfactants preferentially used in the compositions according to the invention are chosen from:
a) nonionic silicone surfactants with an HLB of greater than or equal to 8 at 25° C., used alone or as a mixture; mention may be made especially of:
polydimethylsiloxanes comprising both oxyethylene groups and/or oxypropylene groups. Examples that may be mentioned include the polydimethylsiloxane with oxyethylene/oxypropylene end groups sold as a mixture with caprylic/capric acid triglycerides under the name Abil Care 85 by the company Goldschmidt (INCI name: Bis-PEG/PPG-16/16 PEG/PPG-16/16 dimethicone/caprylic/capric triglyceride), the polydimethylsiloxane containing alpha-omega polyether groups (OE/OP:40/60), sold under the name Abil B8832 by the company Goldschmidt (INCI name: Bis-PEG/PPG-20/20 dimethicone), and the oxyethylenated oxypropylenated polydimethylsiloxane sold under the name Abil B88184 by the company Goldschmidt (INCI name: PEG/PPG-20/6 dimethicone),
the dimethicone copolyol of INCI name PEG/PPG-17/18 dimethicone, such as the product sold under the name Q2-5220 Resin Modifier® by the company Dow Corning, dimethicone copolyol benzoate (Finsolv SLB 1010 and 201© from the company Finetex),
and mixtures thereof;
b) amphoteric silicone surfactants such as dimethicone copolyol phosphates, such as the product sold under the name Pecosil PS100© by the company Phoenix Chemical;
c) and mixtures thereof.

The HLB (hydrophilic-lipophilic balance), in the Griffin sense, is defined in J. Soc. Cosm. Chem. 1954 (volume 5), pages 249-256.

Preferably, the first surfactant is selected from polydimethylsiloxanes comprising both oxyethylene groups and/or oxypropylene groups, dimethicone copolyol, dimethicone copolyol benzoate, dimethicone copolyol phosphates, and mixtures thereof.

According to one preferred mode, the silicone surfactant is chosen from polydimethylsiloxanes comprising both oxyethylene groups and oxypropylene groups, and dimethicone copolyol, and mixtures thereof.

More preferably, the silicone surfactant is chosen from polydimethylsiloxanes comprising both oxyethylene groups and oxypropylene groups.

According to one preferred embodiment of the invention, the silicone surfactant is BIS-PEG/PPG-16/16 PEG/PPG-16/16 DIMETHICONE.

Advantageously, the first surfactant is present in an amount ranging from 0.6 wt. % to 10 wt. %, preferably from 0.8 wt. % to 8 wt. %, more preferably from 0.8 wt. % to 5 wt. %, relative to the total weight of the composition.

The inventors found that with the presence of 0.6 wt. % of silicone surfactant relative to the total weight of the composition of the present invention, the composition does not foam during application.

### Second Surfactants

According to the first aspect, the composition of the present invention comprises at least one second surfactant different from the first surfactant and different from saturated C14-C22 fatty acids.

Preferably, the second surfactant is selected from
mono and polyglyceryl esters of a fatty acid or their ethoxylated derivatives;
esters of polyols with fatty acids with a saturated or unsaturated chain;
sorbitol esters of C8-C24 fatty acids and polyoxyalkylenated derivatives thereof;
esters of fatty acids and glucose or alkylglucose or their ethoxylated derivatives;
sucrose esters;
ethers of a sugar and of C8-C24 fatty alcohols, such as caprylyl/capryl glucoside;
C14-C24 fatty alcohols;
polyoxyethylene alkyl ether carboxylic acids, such as Laureth-5 Carboxylic Acid;
polyoxyethylenated fatty alcohol containing from 6 to 12 oxyethylene units, such as Laureth-9;
polyoxyalkylenated derivative of mono glyceryl ester of a fatty acid such as PEG-20 glyceryl triisostearate,
sarcosinates, such as sodium lauroyl sarcosinate;
fatty acid ester of polyalkylene glycol; and
mixtures thereof.

As polyglyceryl esters of (a) fatty acid(s), mention be made of the product containing 2 to 10 glycerol units, such as polyglyceryl monolaurate, oleate, myristate, caprylate, or stearate comprising 2 to 10 glycerol units, polyglyceryl mono(iso)stearate comprising 2 to 10 glycerol units, polyglyceryl dioleate comprising 2 to 10 glycerol units, polyglyceryl dilaurate comprising 2 to 10 glycerol units, polyglyceryl dimyristate comprising 2 to 10 glycerol units, polyglyceryl trimyristate comprising 2 to 10 glycerol units, polyglyceryl trioleate comprising 2 to 10 glycerol units, and polyglyceryl tricaprylate comprising 2 to 10 glycerol units.

Polyglyceryl esters include, polyglyceryl esters of C16-C22 saturated, unsaturated and branched chain fatty acids, such as polyglyceryl-4 isostearate, polyglyceryl-3 oleate, polyglyceryl-2 oleate, polyglyceryl-2 sesquioleate, triglyceryl diisostearate, diglyceryl monooleate, tetraglyceryl monooleate, or mixtures thereof.

Monoglyceryl esters include, but are not limited to, glyceryl monoesters, preferably glyceryl monoesters of C16-C22 saturated, unsaturated and branched chain fatty acids such as glyceryl oleate, glyceryl monostearate, glyceryl monoisostearate, glyceryl monopalmitate, glyceryl monobehenate, or mixtures thereof.

As mono glyceryl esters of fatty acids, glyceryl stearate (glyceryl mono-, di- and/or tristearate) (CTFA name: glyceryl stearate) or glyceryl ricinoleate or mixtures thereof can be cited, or as polyoxyalkylenated derivatives thereof, mono-, di- or triester of fatty acids with a polyoxyalkylenated glycerol (mono-, di- or triester of fatty acids with a polyalkylene glycol ether of glycerol), preferably polyoxyethylenated glyceryl stearate (mono-, di- and/or tristearate), such as PEG-20 glyceryl stearate (mono-, di-, tristearate and/or triisostearate) can be cited. Preferably, the polyoxyalkylenated derivative of mono glyceryl ester of fatty acids includes 10 to 40 oxyethylene units, such as PEG-20 glyceryl triisostearate.

The second surfactant may be selected from esters of polyols with fatty acids with a saturated or unsaturated chain containing for example from 8 to 24 carbon atoms, preferably 12 to 22 carbon atoms, or polyoxyalkylenated derivatives thereof, preferably containing from 10 to 200, or more preferably from 10 to 100 oxyalkylene units, such as mono glyceryl esters or poly glyceryl esters of a C8-C24, preferably C12-C22, fatty acid and polyoxyalkylenated derivatives thereof, preferably containing from 10 to 200, or more preferably from 10 to 100 oxyalkylene units; sorbitol esters of a C8-C24, preferably C12-C22, fatty acid or polyoxyalkylenated derivatives thereof, preferably containing from 10 to 200, or more preferably from 10 to 100 oxyalkylene units; sugar (sucrose, maltose, glucose, fructose, and/or alkylglycose) esters of a C8-C24, preferably C12-C22, fatty acid or polyoxyalkylenated derivatives thereof, preferably containing from 10 to 200, or more preferably from 10 to 100 oxyalkylene units; ethers of fatty alcohols; ethers of sugar and a C8-C24, preferably C12-C22, fatty alcohol or alcohols; or mixtures thereof.

Mixtures of these surfactants, such as for example the product containing glyceryl stearate and PEG-100 stearate, marketed under the name ARLACEL 165 by CRODA, and the product containing glyceryl stearate (glyceryl mono- and distearate) and potassium stearate marketed under the name TEGIN by Goldschmidt (CTFA name: glyceryl stearate SE), can also be used.

The sorbitol esters of C8-C24 fatty acids and polyoxyalkylenated derivatives thereof can be selected from sorbitan palmitate, sorbitan isostearate, sorbitan trioleate and esters of fatty acids and alkoxylated sorbitan containing for example from 20 to 100 EO, such as for example sorbitan monostearate (CTFA name: sorbitan stearate), sold by the company ICI under the name Span 60, sorbitan monopalmitate (CTFA name: sorbitan palmitate), sold by the company ICI under the name Span 40, or sorbitan tristearate 20 EO (CTFA name: polysorbate 65), sold by the company ICI under the name Tween 65, polyethylene sorbitan trioleate (polysorbate 85) or the compounds marketed under the trade names Tween 20 or Tween 60 by Uniqema.

As esters of fatty acids and glucose or alkylglucose, glucose palmitate, alkylglucose sesquistearates such as methylglucose sesquistearate, alkylglucose palmitates such as methylglucose or ethylglucose palmitate, methylglucoside fatty esters, the diester of methylglucoside and oleic acid (CTFA name: Methyl glucose dioleate), the mixed ester of methylglucoside and the mixture of oleic acid/hydroxystearic acid (CTFA name: Methyl glucose dioleate/hydroxystearate), the ester of methylglucoside and isostearic acid (CTFA name: Methyl glucose isostearate), the ester of methylglucoside and lauric acid (CTFA name: Methyl glucose laurate), the mixture of monoester and diester of methylglucoside and isostearic acid (CTFA name: Methyl glucose sesqui-isostearate), the mixture of monoester and diester of methylglucoside and stearic acid (CTFA name: Methyl glucose sesquistearate) and in particular the product marketed under the name Glucate SS by AMERCHOL, and mixtures thereof can be cited.

As ethoxylated ethers of fatty acids and glucose or alkylglucose, ethoxylated ethers of fatty acids and methylglucose, and in particular the polyethylene glycol ether of the diester of methylglucose and stearic acid with about 20 moles of ethylene oxide (CTFA name: PEG-20 methyl glucose distearate) such as the product marketed under the name Glucam E-20 distearate by AMERCHOL, the polyethylene glycol ether of the mixture of monoester and diester of methyl-glucose and stearic acid with about 20 moles of ethylene oxide (CTFA name: PEG-20 methyl glucose sesquistearate) and in particular the product marketed under the name Glucamate SSE-20 by AMERCHOL and that marketed under the name Grillocose PSE-20 by GOLDSCHMIDT, or mixtures thereof, can for example be cited.

As sucrose esters, saccharose palmito-stearate, saccharose stearate and saccharose monolaurate can for example be cited.

As sugar ethers, alkylpolyglucosides can be used, and for example, ethers of a sugar and of C₈-C₂₄ fatty alcohols including decylglucoside such as the product marketed under the name MYDOL 10 by Kao Chemicals, the product marketed under the name PLANTAREN 2000 by Henkel, and the product marketed under the name ORAMIX NS 10 by Seppic, caprylyl/capryl glucoside such as the product marketed under the name ORAMIX CG 110 by Seppic or under the name LUTENSOL GD 70 by BASF, laurylglucoside such as the products marketed under the names PLANTAREN 1200 N and PLANTACARE 1200 by Henkel, coco-glucoside such as the product marketed under the name PLANTACARE 818/UP by Henkel, cetostearyl glucoside possibly mixed with cetostearyl alcohol, marketed for example under the name MONTANOV 68 by Seppic, under the name TEGO-CARE CG90 by Goldschmidt and under the name EMULGADE KE3302 by Henkel, arachidyl glucoside, for example in the form of the mixture of arachidyl and behenyl alcohols and arachidyl glucoside marketed under the name MONTANOV 202 by Seppic, cocoylethylglucoside, for example in the form of the mixture (35/65) with cetyl and stearyl alcohols, marketed under the name MONTANOV 82 by Seppic, or mixtures thereof can in particular be cited.

As C14-C24 fatty alcohols, mention can be made of stearyl alcohol, cetyl alcohol, behenyl alcohol, arachidyl alcohol, lignocerylic alcohol and mixtures thereof.

Preferably, the fatty acid ester of polyalkylene glycol is an ester of polyethylene glycol comprising from 1 to 200 oxyethylene groups and of a saturated or unsaturated fatty acid comprising from 12 to 30 carbon atoms, more preferably, the fatty acid ester of polyalkylene glycol is selected from polyethylene glycol 2 OE monostearate or polyethylene glycol 100 monostearate (PEG-100 stearate).

Most preferably, the second surfactant is selected from mono- and polyglyceryl esters of a fatty acid, ethers of a sugar and of C8-C24 fatty alcohols, fatty acid esters of polyalkylene glycol, C14-C24 fatty alcohols, and mixtures thereof.

Advantageously, the second surfactant is selected from glyceryl stearate, PEG-100 stearate, arachidyl alcohol, behenyl alcohol, arachidyl glucoside, and mixtures thereof.

Advantageously, the second surfactant is present in an amount ranging from 0.2 wt. % to 20 wt. %, preferably from 0.4 wt. % to 15 wt. %, more preferably from 0.8 wt. % to 12 wt. %, relative to the total weight of the composition.

### Structuring Agents

According to the first aspect, the composition of the present invention comprises at least one structuring agent selected from saturated C14-C22 fatty acids.

As examples of saturated C14-C22 fatty acid, mention can be made of myristic acid, pentadecanoic acid, palmitic acid, heptadecanoic acid, stearic acid, cetearylic acid, arachidic acid, behenic acid, and a combination thereof.

Preferably, the structuring agent is selected from saturated C14-C18 fatty acids.

More preferably, the structuring agent is selected from linear and saturated C14-C18 fatty acids.

Particularly preferably, the structuring agent is selected from myristic acid, palmitic acid, stearic acid, and a combination thereof.

Advantageously, the structuring agent is present in an amount ranging from 0.5 wt. % to 30 wt. %, preferably from 1 wt. % to 20 wt. %, more preferably from 1.3 wt. % to 10 wt. %, most preferably from 1.3 wt. % to 6 wt. %, relative to the total weight of the composition.

### Saponifiers

According to the first aspect, the composition of the present invention comprises at least one saponifier.

The saponifier can be used to react with a saturated C14-C22 fatty acid to produce a salt.

The saponifier can be, for example, inorganic bases, such as alkali metal hydroxides (for example, sodium hydroxide and potassium hydroxide), alkaline earth metal hydroxides (for example, magnesium hydroxide) or ammonium hydroxide, or organic bases, for example, monoethanolamine (MEA), diethanolamine, triethanolamine, tromethamine, N-methylglucamine, arginine, lysine and arginine.

Mention can be also made of sodium carbonate, potassium carbonate, sodium bicarbonate, and potassium bicarbonate.

Preferably, the saponifier is selected from sodium hydroxide, potassium hydroxide, magnesium hydroxide, ammonium hydroxide, monoethanolamine, diethanolamine, triethanolamine, tromethamine, N-methylglucamine, arginine, lysine, arginine, and mixtures thereof.

Advantageously, the saponifier is present in an amount ranging from 0.05 wt. % to 2 wt. %, preferably from 0.1 wt. % to 0.8 wt. %, more preferably from 0.15 wt. % to 0.6 wt. %, relative to the total weight of the composition.

Advantageously, the weight ratio of the structuring agent to the second surfactant is from 0.2 to 2.0, preferably from 0.4 to 1.5.

### Aqueous Phase

As an oil-in-water emulsion, the cosmetic composition of the present invention comprises a continuous aqueous phase.

Said aqueous phase comprises water.

Preferably, the continuous aqueous phase comprises an organic solvent miscible with water (at room temperature 25° C.) such as glycerin and glycols having from 2 to 20 carbon atoms, preferably from 2 to 10 carbon atoms, and preferentially having from 2 to 6 carbon atoms, such as propylene glycol, butylene glycol, pentylene glycol, hexylene glycol, caprylyl glycol, dipropylene glycol, diethylene glycol; and mixtures thereof.

Advantageously, said aqueous phase is present in an amount ranging from 30 wt. % to 95 wt. %, preferably from 35 wt. % to 90 wt. %, and more preferably from 40 wt. % to 85 wt. % of the total weight of the composition.

### Fatty Phase

As an oil-in-water emulsion, the composition of the present invention comprises at least one dispersed oily phase.

Said fatty phase preferably comprises at least one oil.

The oil can be volatile or non-volatile.

The term “oil” means a water-immiscible non-aqueous compound that is liquid at room temperature (25° C.) and at atmospheric pressure (760 mmHg). The term “non-volatile oil” means an oil that may remain on keratin materials at room temperature and atmospheric pressure for at least several hours and that especially has a vapour pressure of less than 10-3 mmHg (0.13 Pa). A non-volatile oil may also be defined as having an evaporation rate such that, under the conditions defined previously, the amount evaporated after 30 minutes is less than 0.07 mg/cm².

These oils may be of plant, mineral or synthetic origin.

Said oil can be selected from hydrocarbonated, silicone or fluorinated oils.

The term “hydrocarbon-based oil” means an oil formed essentially from, or even constituted by, carbon and hydrogen atoms, and optionally O and N atoms, and free of Si and F heteroatoms. Such oil can contain alcohol, ester, ether, carboxylic acid, amine and/or amide groups.

The term “silicone oil” means an oil containing at least one silicon atom, especially containing Si—O groups.

The term “fluorinated oil” means an oil containing at least one fluorine atom.

Preferably, the oil is selected from hydrocarbon-based oils, such as isohexadecane, squalane, caprylic/capric triglyceride, isononyl isononanoate, pentaerythrityl tetraisostearate, myristyl myristate, and silicone oils, such as dimethicone.

The fatty phase can also comprise a non-oil fatty substance, for example butters and waxes, such as butyrospermum parkii butter, cera alba, and beeswax, so as to build texture and deliver a suitable skin finish.

Advantageously, the fatty phase is present in an amount ranging from 2 wt. % to 40 wt. %, preferably from 8 wt. % to 35 wt. %, more preferably from 10 wt. % to 30 wt. %, relative to the total weight of the composition of the present invention.

### Cosmetic Active Ingredients

Preferably, the composition of the present invention comprises a cosmetic active ingredient.

As examples of cosmetic active ingredient, mention can be made of natural extracts; vitamins such as vitamin A (retinol), vitamin E (tocopherol), vitamin C (ascorbic acid), vitamin B5 (panthenol), vitamin B3 (niacinamide), and derivatives of said vitamins (in particular esters) and mixtures thereof; urea; caffeine; C-glycosides such as hydroxypropyl tetrahydropyrantriol; salicylic acid and derivatives thereof; alpha-hydroxyacids such as lactic acid or glycolic acid and derivatives thereof; sunscreens; extracts from algae, fungi, plants, yeasts and bacteria; enzymes; moisturing agents such as sodium hyaluronate and sodium acetylated hyaluronate, agents acting on the microcirculation, and mixtures thereof.

It is easy for the skilled in the art to adjust the amount of the additional cosmetic active ingredient based on the final use of the composition according to the present invention.

### Additional Adjuvants or Additives

The composition of the present invention may comprise conventional cosmetic adjuvants or additives, for instance fragrances, preserving agents (for example, chlorphenesin and phenoxyethanol) and bactericides, thickeners, pH regulators, and mixtures thereof.

The skilled in the art can select the amount of the additional adjuvants or additive so as not to adversely impact the final use of the composition according to the present invention.

According to a particularly preferred embodiment, the present invention provides a composition in the form of an oil-in-water emulsion for caring for and/or making up keratin materials comprising, relative to the total weight of the composition:
(i) from 0.8 wt. % to 5 wt. % of at least one first surfactant selected from polydimethylsiloxanes comprising both oxyethylene groups and oxypropylene groups,
(ii) from 0.8 wt. % to 12 wt. % of at least one second surfactant selected from mono- and polyglyceryl esters of a fatty acid, ethers of a sugar and of C8-C24 fatty alcohols, fatty acid esters of polyalkylene glycol, C14-C24 fatty alcohols, and mixtures thereof; and
(iii) from 1.3 wt. % to 6 wt. % of at least one structuring agent selected from linear and saturated C14-C18 fatty acids; and
(iv) from 0.15 wt. % to 0.6 wt. % of at least one saponifier selected from sodium hydroxide, potassium hydroxide, magnesium hydroxide, ammonium hydroxide, monoethanolamine, diethanolamine, triethanolamine, tromethamine, N-methylglucamine, arginine, lysine, arginine, and a combination thereof,
wherein the weight ratio of the structuring agent to the second surfactant is from 0.2 to 2.0.

### Galenic Form and Method

The composition of the present invention is in the form of oil-in-water emulsion.

The composition according to the present invention has a lamellar structure.

The term “lamellar structure” means a liquid crystal structure, or a swollen or non-swollen crystalline lamellar hydrate phase with plane symmetry, comprising several amphiphilic bilayers arranged in parallel and separated by a liquid medium which is generally water.

The composition of the present invention can be used for caring for and/or making up keratin materials.

According to the second aspect, the present invention provides a non-therapeutic method for caring for and/or making up keratin materials, comprising applying the composition according to the first aspect of the present invention to the keratin materials.

The following examples serve to illustrate the present invention without, however, being limiting in nature.

### EXAMPLES

Main raw materials used, trade names and supplier thereof are listed in Table 1.

TABLE 1

| INCI Name | Trade Name | Supplier |
|---|---|---|
| GLYCERYL STEARATE | ARLACEL ™ 165 | CRODA |
| (and) PEG-100 | | |
| STEARATE | | |
| SODIUM | COVACRYL ® | SENSIENT |
| POLYACRYLATE | MV60 | |
| STEARIC ACID | PALMAC ® | IOI GROUP |
| | 50-18/MB | OLEOCHEMICALS |
| POTASSIUM | LESSIVE DE | QUARON |
| HYDROXIDE | POTASSE 50% | |
| ARACHIDYL ALCOHOL | MONTANOV 202 | SEPPIC |
| (and) BEHENYL | | |
| ALCOHOL (and) | | |
| ARACHIDYL | | |
| GLUCOSIDE | | |
| BUTYROSPERMUM | FAIR FOR LIFE | OLVEA |
| PARKII BUTTER | REFINED SHEA | |
| | BUTTER | |
| ISONONYL | ERCAREL ISN/O | ERCA |
| ISONONANOATE | | |
| PENTAERYTHRITYL | JOLEE 7181 | OLEON |
| TETRAISOSTEARATE | | |
| MYRISTYL MYRISTATE | TEGOSOFT ® MM | EVONIK |
| | | GOLDSCHMIDT |
| CHLORPHENESIN | MACROCIDE-OL | MACROCARE |
| BEESWAX | CERABEIL LOR | BAERLOCHER |
| | | (CERESINE) |
| XANTHAN GUM | KELTROL CG-T | CP KELCO |
| DIMETHICONE | DOWSIL ™ SH | DOW CORNING |
| | 200 Fluid 5 CST | (DOW CHEMICAL) |
| HYDROXYPROPYL | MEXORYL SCS | CHIMEX (NOVEAL) |
| TETRAHYDRO- | | |
| PYRANTRIOL | | |
| BIS-PEG/PPG-16/16 | ABIL CARE 85 | EVONIK |
| PEG/PPG-16/16 | | GOLDSCHMIDT |
| DIMETHICONE (and) | | |
| CAPRYLIC/CAPRIC | | |
| TRIGLYCERIDE | | |

### Invention Examples 1-3 and Comparative Examples 1-2

Compositions of invention examples (IE.) 1-3 and comparative examples (CE.) 1-2 were prepared according to the amounts given in Table 2. The amount of each component is given in % by weight of the total weight of the composition containing it.

TABLE 2

| Phase | INCI US | CE. 1 | CE. 2 | IE. 1 | IE. 2 | IE. 3 |
|---|---|---|---|---|---|---|
| A | POTASSIUM HYDROXIDE | 0.28 | 0.28 | 0.28 | 0.12 | 0.20 |
| | WATER | To | To | To | To | To |
| | | 100 | 100 | 100 | 100 | 100 |
| | GLYCERIN | 8.00 | 8.00 | 8.00 | 8.00 | 7.00 |
| | CHLORPHENESIN | 0.20 | 0.20 | 0.20 | 0.20 | 0.20 |
| | CAPRYLYL GLYCOL | 0.30 | 0.30 | 0.30 | 0.30 | 0.30 |
| | PENTYLENE GLYCOL | 1.50 | 1.50 | 1.50 | 1.50 | 1.50 |
| B | STEARIC ACID | 3.00 | 3.00 | 3.00 | 3.00 | 1.50 |
| | GLYCERYL STEARATE (and) PEG- | 1.60 | 1.60 | 1.60 | 0.80 | 0.80 |
| | 100 STEARATE¹⁾ | | | | | |
| | BEESWAX | 2.73 | 2.73 | 2.73 | 2.73 | 2.40 |
| | BUTYROSPERMUM PARKII | 3.55 | 3.55 | 3.55 | 3.55 | 3.10 |
| | (SHEA) BUTTER | | | | | |
| | ISONONYL ISONONANOATE | 1.00 | 1.00 | 1.00 | 1.00 | — |
| | PENTAERYTHRITYL | 1.00 | 1.00 | 1.00 | 1.00 | 0.40 |
| | TETRAISOSTEARATE | | | | | |
| | ARACHIDYL ALCOHOL (and) | 2.00 | 2.00 | 2.00 | 2.00 | 2.00 |
| | BEHENYL ALCOHOL (and) | | | | | |
| | ARACHIDYL GLUCOSIDE²⁾ | | | | | |
| | BIS-PEG/PPG-16/16 PEG/PPG- | / | 0.45 | 0.90 | 0.90 | 0.80 |
| | 16/16 DIMETHICONE (and) | | | | | |
| | CAPRYLIC/CAPRIC | | | | | |
| | TRIGLYCERIDE³⁾ | | | | | |
| | MYRISTYL MYRISTATE | 1.23 | 1.23 | 1.23 | 1.23 | 1.10 |
| C | XANTHAN GUM | 0.25 | 0.25 | 0.25 | 0.25 | 0.25 |
| | DIMETHICONE | 2.50 | 2.50 | 2.50 | 2.50 | 1.00 |
| | SODIUM POLYACRYLATE | 0.80 | 0.80 | 0.80 | 0.80 | 0.80 |
| D | HYDROXYPROPYL | 20.00 | 20.00 | 20.00 | 20.00 | 20.00 |
| | TETRAHYDROPYRANTRIOL⁴⁾ | | | | | |

| | | | | | |
|---|---|---|---|---|---|
| Weigh ratio of fatty | 0.83 | 0.83 | 0.83 | 1.07 | 0.54 |
| acid/the second surfactant* | | | | | |

GLYCERYL STEARATE (and) PEG-100 STEARATE¹⁾comprising 50 wt. % of GLYCERYL STEARATE and 50 wt. % of PEG-100 STEARATE.
ARACHIDYL ALCOHOL (and) BEHENYL ALCOHOL (and) ARACHIDYL GLUCOSIDE ²⁾comprising 55 wt. % of ARACHIDYL ALCOHOL, 30 wt. % of BEHENYL ALCOHOL and 15 wt. % of ARACHIDYL GLUCOSIDE.
BIS-PEG/PPG-16/16 PEG/PPG-16/16 DIMETHICONE (and) CAPRYLIC/CAPRIC TRIGLYCERIDE³⁾comprising 87 wt. % of BIS-PEG/PPG-16/16 PEG/PPG-16/16 DIMETHICONE and 12.98 wt. % of CAPRYLIC/CAPRIC TRIGLYCERIDE.
HYDROXYPROPYL TETRAHYDROPYRANTRIOL⁴⁾is HYDROXYPROPYL TETRAHYDROPYRANTRIOL in water and propylene glycol.at a content of 70 wt. %, relative to the total weight of HYDROXYPROPYL TETRAHYDROPYRANTRIOL and water and propylene glycol.
*The second surfactant comprises GLYCERYL STEARATE (and) PEG-100 STEARATE and ARACHIDYL ALCOHOL (and) BEHENYL ALCOHOL (and) ARACHIDYL GLUCOSIDE.

Compositions of invention examples 1-3 are compositions according to the present invention.

Composition of comparative example 1 does not comprise a silicone surfactant.

Composition of comparative example 2 comprises 0.45 wt. % of silicone surfactant, relative to the total weight of the composition.

### Preparation Process:

The compositions listed above were prepared as follows:
1) weighting the ingredients for Phase A into a container and mixing them to obtain a transparent mixture, then stirring at 75° C. under a stirring rate of 500 rpm until a yellowish transparent liquid was obtained;
2) heating the Phase B premix at 85° C., then adding it to the yellowish transparent liquid at 75° C. under a stirring rate of 1500 rpm and mixing for 10 minutes to obtain a white emulsion;
3) adding the phase C premix at 75° C. under a stirring rate of 1500 rpm to obtain a white emulsion;
4) adding the phase D at 70° C. under a stirring rate of 1500 rpm to obtain a white thin cream, then cooling down to room temperature with stirring to obtain the composition.

### Evaluation

Each composition of invention examples 1-3 and comparative examples 1-2 was evaluated in terms of lamellar structure, foaming and stability.

### Lamellar Structure

Whether a lamellar structure is formed was evaluated as follows.

Compositions was observed using a Leica DLMB microscope under 90° cross polarized light, and microscopic pictures were taken. If characteristic optical effects of lamellar structure were observed, it means that lamellar structure was formed.

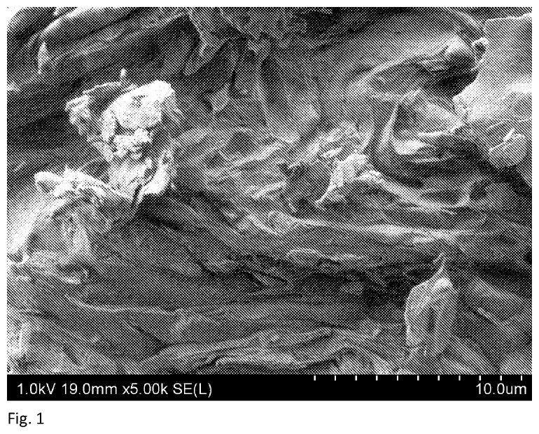

FIG. 1 shows a photo of the composition of invention example 1 (IE.1) taken with a polarized light microscopy.

It can be seen from FIG. 1 that a lamellar structure is formed in the compositions of invention example 1.

Similar photos were also obtained for the compositions of invention examples 2-3 (IE.2-3) and comparative examples 1-2.

The results of lamellar structure of each composition prepared above were listed in Table 3.

TABLE 3

| Item | CE. 1 | CE. 2 | IE. 1 | IE. 2 | IE. 3 |
|---|---|---|---|---|---|
| lamellar structure | YES | YES | YES | YES | YES |

### Foaming

The compositions prepared above were applied on a hand to observe if there is foaming during application.

The results on foaming were summarized in Table 4.

TABLE 4

| Formula | CE. 1 | CE. 2 | IE. 1 | IE. 2 | IE. 3 |
|---|---|---|---|---|---|
| Foaming | Yes | A little | No | No | No |
| | | foaming | | | |

### Stability

The stability was evaluated as follows:

The appearance of each composition prepared above was observed immediately after it was prepared at room temperature or upon storage at 45° C. for 2 months.

If there is wax recrystallization at room temperature or 45° C., then the stability test fails, and there is no wax recrystallization at both room temperature and 45° C., then the stability test pass.

The results on stability were summarized in Table 5.

TABLE 5

| Formula | | CE. 1 | CE. 2 | IE. 1 | IE. 2 | IE. 3 |
|---|---|---|---|---|---|---|
| Appearance | RT T0* | No wax | No wax | No wax | No wax | No wax |
| | 45° C. T2M** | No wax | No wax | No wax | No wax | No wax |

| | | | | | |
|---|---|---|---|---|---|
| Stability | Pass | Pass | Pass | Pass | Pass |

RT T0* immediately after the composition to be tested was prepared.
45° C. T2M** upon storage at 45° C. for 2 months.

It can be seen that compositions of invention examples 1-3 are stable and do not foam during application.

1: A composition in the form of an oil-in-water emulsion for caring for and/or making up keratin materials, comprising:
(i) relative to the total weight of the composition, at least 0.6 wt. % of at least one first surfactant selected from silicone surfactants;
(ii) at least one second surfactant different from the first surfactant and different from saturated C14-C22 fatty acids;
(iii) at least one structuring agent selected from saturated C14-C22 fatty acids; and
(iv) at least one saponifier,
wherein a weight ratio of the structuring agent to the second surfactant is from 0.2 to 2.0.

2: The composition according to claim 1, wherein the first surfactant is selected from the group consisting off polydimethylsiloxanes comprising oxyethylene groups and/or oxypropylene groups, dimethicone copolyol, dimethicone copolyol benzoate, dimethicone copolyol phosphates, and mixtures thereof.

3: The composition according to claim 1, wherein the first surfactant is present in an amount ranging from 0.6 wt. % to 10 wt. % relative to the total weight of the composition.

4: The composition according to claim 1, wherein the second surfactant is selected from the group consisting of
mono and polyglyceryl esters of a fatty acid or their ethoxylated derivatives;
esters of polyols with fatty acids with a saturated or unsaturated chain;
sorbitol esters of C8-C24 fatty acids and polyoxyalkylenated derivatives thereof;
esters of fatty acids and glucose or alkylglucose or their ethoxylated derivatives;
sucrose esters;
ethers of a sugar and of C8-C24 fatty alcohols;
C14-C24 fatty alcohols;
polyoxyethylene alkyl ether carboxylic acids;
polyoxyethylenated fatty alcohol containing from 6 to 12 oxyethylene units;
polyoxyalkylenated derivative of mono glyceryl ester of a fatty acid,
sarcosinates;
fatty acid ester of polyalkylene glycol; and
mixtures thereof.

5: The composition according to claim 1, wherein the second surfactant is present in an amount ranging from 0.2 wt. % to 20 wt. % relative to the total weight of the composition.

6: The composition according to claim 1, wherein the structuring agent is selected from linear and saturated C14-C18 fatty acids and a combination thereof.

7: The composition according to claim 1, wherein the structuring agent is present in an amount ranging from 0.5 wt. % to 30 wt. % relative to the total weight of the composition.

8: The composition according to claim 1, wherein the saponifier is selected from the group consisting of sodium hydroxide, potassium hydroxide, magnesium hydroxide, ammonium hydroxide, monoethanolamine, diethanolamine, triethanolamine, tromethamine, N-methylglucamine, arginine, lysine, arginine, and a combination thereof.

9: The composition according to claim 1, wherein the saponifier is present in an amount ranging from 0.05 wt. % to 2 wt. % relative to the total weight of the composition.

10: The composition according to claim 1, comprising, relative to the total weight of the composition:
(i) from 0.8 wt. % to 5 wt. % of at least one first surfactant selected from the group consisting of polydimethylsiloxanes comprising both oxyethylene groups and oxypropylene groups,
(ii) from 0.8 wt. % to 12 wt. % of at least one second surfactant selected from the group consisting of mono- and polyglyceryl esters of a fatty acid, ethers of a sugar and of C8-C24 fatty alcohols, fatty acid esters of polyalkylene glycol, C14-C24 fatty alcohols, and mixtures thereof; and
(iii) from 1.3 wt. % to 6 wt. % of at least one structuring agent selected from the group consisting of linear and saturated C14-C18 fatty acids; and
(iv) from 0.15 wt. % to 0.6 wt. % of at least one saponifier selected from the group consisting of sodium hydroxide, potassium hydroxide, magnesium hydroxide, ammonium hydroxide, monoethanolamine, diethanolamine, triethanolamine, tromethamine, N-methylglucamine, arginine, lysine, arginine, and a combination thereof.

11: A non-therapeutic method for caring for keratin materials, comprising applying the composition according to claim 1 to the keratin materials.
